(19) United States
(12) Patent Application Publication
(10) Pub. No.: US 2008/0131902 A1
(43) Pub. Date: Jun. 5, 2008

(54) Personalized skin care composition and method for production thereof

(75) Inventors: Ze'ev MAOR, Dead Sea (IL); Yoram MILNER, Mevaseret Zion (IL); Francois Menachem BREGEGER, Jerusalem (IL)

Correspondence Address: BACON & THOMAS, PLLC, 625 SLATERS LANE, FOURTH FLOOR, ALEXANDRIA, VA, 22314, US

(73) Assignee: AHAVA-DEAD SEA LABORATORIES LTD., Mizpe Shalem (IL)

(21) Appl. No.: 11/984,456

(22) Filed: Nov. 19, 2007

Related U.S. Application Data
(63) Continuation-in-part of application No. 10/934,441, filed on Sep. 7, 2004, now abandoned.

(30) Foreign Application Priority Data
Sep. 7, 2003 (IL) 157786

(51) Int. Cl. C12Q 1/68; C12Q 1/02; G01N 33/53; C12Q 1/34; C12Q 1/37; C12Q 1/48

(52) U.S. Cl. 435/6; 435/29; 435/7.92; 435/18; 435/24; 435/15

(57) ABSTRACT

A method for providing a personalized skin-care composition for an individual includes sampling the skin of an individual for whom a personalized skin-care composition is to be provided, testing for a predetermined set of biomarkers so as to produce an individual biomarker profile, analyzing the biomarker profile using a biomarker profile database, and producing a personalized skin-care composition based on the results. The personalized skin-care composition is adapted for treating one or more skin conditions which the individual has, and can be adapted according to the preferences of the individual.

### CROSS REFERENCE TO OTHER APPLICATIONS

The present invention is a continuation-in-part of U.S. patent application Ser. No. 10/934,441, filed Sep. 7, 2004.

### FIELD OF THE INVENTION

The present invention relates generally to the field of health care products. More specifically, the present invention relates to personalized skin care products and to a method for their production.

### BACKGROUND OF THE INVENTION

The skin is the largest organ of the human body and serves several vital functions, including body protection, absorption, secretion, excretion, thermo-regulation, pigment synthesis, sensory perception and immunity. It is constantly exposed to harmful environments and extreme conditions, such as ultra-violet radiation, urban pollution, industrial contamination, pathogenic attacks, allergenic challenges, carcinogenic substances, etc. . . . Hence, the skin regularly needs topical treatment, which is usually provided by dermo-cosmetics, i.e., cosmetic preparations and/or dermal pharmaceutics. The dermo-cosmetics are targeted to achieve effective skin protection or prevention of skin ailments, by eliminating or avoiding skin sensitivity responses, skin irritation, skin psoriasis, dryness or sun burn, by controlling or delaying skin aging, appearance of wrinkles, decreased skin smoothness, decreased general appearance (e.g., loss of healthy glow etc).

Commercially available dermo-cosmetics contain effective active agents, formulated in various substances (gels, creams, lotions, masks etc.) and are usually made of high-quality ingredients. Nevertheless, it has been shown that due to relatively poor delivery characteristics of dermo-cosmetics vehicles, only a small amount of the active ingredients actually reaches the relevant skin cells where therapeutic treatment is needed. Moreover, some users are sensitive to dermo-cosmetics preparations and tend to develop signs of irritability and toxicity following application of those chemicals. In many other cases, the skin is not affected at all by the dermal treatment.

Currently, it is generally the individuals themselves who make the correlation between their skin condition and the kind of dermo-cosmetic composition they need, as a prior step before formal treatment. Users typically chose the desired product themselves “off the shelf” according to their limited understanding. Such purchases are extensively affected both by ever-changing trends and by exposure of users to advertisements. In some cases, the user consults the employee, who stands at the point of sale. Usually, this promotional-marketing worker is not an expert on skin conditions. In other cases, cosmeticians examine the skin to be treated visually and subsequently, according to their accumulated Less commonly, a physician e.g., a dermatologist, studies the topical appearance of the skin and gives professional advice to the user on what treatment is appropriate.

Various skin-sampling techniques have been disclosed in the literature. A dermatological punch biopsy is usually performed by means of a round knife ranging from 2 to 10 mm in size. A plurality of 5 mm punches generally provides adequate epidermis samples for analysis (See for example Zuber, T. J., 2002. Am. Fam. Physician 65, 1155-1164). Another epidermal sampling is denoted as ‘tape stripping’ and was introduced by Mattin et al., 1996. Skin Pharmacol. 9, 69-77, and by others. Suction blistering is an ex vivo sampling technique that was suggested by Falabella, R., 2000. Int. J. of Dermatol. 39, 670-672. According to this method, metal cups are attached to the skin surface of each forearm. Skin is suctioned using a vacuum pump with negative pressure of 150 mm Hg. Epidermal blisters, 1.6 cm in diameter, are induced after 2-5 hours of suction. Further epidermis sampling methods were suggested by Dimri, G. P. et al., 1995, Proc. Natl. Acad. Sci. USA 92, 9363-9367 and by others.

Some molecular approaches have been suggested for diagnosing or treating skin conditions. Among those suggested, U.S. Pat. Application No. 2002/0034741 to Werner teaches the use of polypeptides or nucleic acids, encoded to create a gene family for the diagnosis or treatment of skin or intestinal disorders, and their use for the identification of pharmacologically active substances.

Similarly, U.S. Pat. Application No. 2002/0012927 to Burmer discloses nucleic acids and proteins important in cell proliferation and senescence, associated with the aging process, and in particular with skin aging. A cosmetic composition containing those nucleic acids and proteins could inhibit skin cell aging in a patient.

It is obvious from the state of the art that various skin-sampling methods are known, and in fact, many continue to be developed. Moreover, a variety of different skin treatments are continuously being developed. However, each skin treatment, such as the one described in Burner, is designed to address only a specific skin condition or group of symptoms associated with a specific condition (i.e., skin aging).

There has been a long-felt need for a skin-care product that treats most or all of the skin conditions which an individual suffers from, instead of just addressing one of them. Such a product would be custom-made and “all-in-one”, according to the individual skin profile of the user (i.e., client). Such a product would eliminate the need for a user to purchase a separate product for treating each of his different skin conditions, and allow him to avoid the confusion as to which products to use and which are appropriate for use for his specific skin and which are not. Finally, such a product and skin-care service would be readily available to the general public, thus providing a therapeutic, health-promoting product, without necessitating dermatologists' visits or complicated, invasive skin testing methods.

These and other features are the goals of the present invention, and they will become more readily apparent from the summary of the invention and the detailed description of the invention that follows.

### SUMMARY OF THE INVENTION

The present invention relates to a novel method for providing individuals with personalized skin-care products. The goal of the invention is to provide a beauty and health-care service, in which the specific skin profile of an individual is used for providing a personalized dermo-cosmetic composition. The product provided, which may be, for example, a cream, a gel, or emulsion, is designed specifically according to specific skin profile of the individual. Furthermore, the product may be adapted according to the individual's preferences, such as with a certain aroma or aromas, or with all organic ingredients. The strength of the composition may also be determined according to the preferences of the individual.

It will be appreciated that the present invention is designed with the general public in mind. The skin is one of the largest organs of the body and it serves to form a protective barrier for the body from the outside environment. The skin is also a very distinct organ because every individual has unique skin: some have more sensitive skin, some are more prone to acne, some may have mild or more serious forms of other skin conditions such as psoriasis, allergies, low pH, etc. . . . Most individuals have skin conditions which fall within the range of “normal” skin. Individuals only seek the treatment of a dermatologist when a particular skin condition worsens such that the individual is suffering from it and the condition now presents a handicap. In such cases, the individual seeks treatment of a dermatologist or other doctor, and a specific, strong, medication is provided to try to heal or reduce the severity of the particular condition. The present invention is not intended for such cases. Rather, the present invention is intended for providing a service to the general public, for the maintenance and enhancement of the health of the skin. Thus, the dermo-cosmetic compositions of the present invention do not treat only one specific condition. They are instead designed to provide treatment for a wide range of skin conditions or dispositions, according to the specific skin profile of the client. They also designed with the client's individual preferences in mind, a feature not currently available using traditional medications.

Thus, the present invention relates a skin-care service for enabling health and beauty-conscious individuals to obtain a personalized skin-care product, developed according to their particular skin profile and preferences. Skin testing is performed in a non-invasive or minimally invasive manner. The service is not meant to be provided at the doctor's office (though it may in fact be), since individuals who visit dermatologists are usually those who have skin diseases that are outside of the normal “healthy” range. Rather, the service may be provided at the make-up counter in a mall, for example, or by a cosmetician.

The method of the present invention utilizes only approved ingredients for treating hypodermic conditions. The product provided is a holistic product for the individual being treated. It is not aimed at the treatment of one specific skin condition, but rather, at the treatment of the whole skin condition, based on a wide range of parameters.

The present invention thus relates to a method for providing a personalized skin-care composition for an individual, comprising;

(a) sampling the skin of an individual for whom a personalized skin-care composition is to be provided;

(b) testing for a predetermined set of biomarkers so as to produce an individual biomarker profile;

(c) analyzing said biomarker profile using a biomarker profile database;

(d) producing a personalized skin-care product based on the results of step (c);

wherein the personalized skin-care composition is adapted for treating one or more skin conditions which the individual has.

In preferred embodiments of the present invention, an exhaustive questionnaire aimed to determine all useful knowledge relevant to a patient's particular skin condition, usual dermatological treatment, cosmetic regular skin care, general health status, medical antecedents and heredity, life habits, etc. . . . is completed by the patient and/or medical practitioner This data will be registered in a personal file, together with biological measurements, in order to analyze their correlations and further introduced into the data base system.

According to preferred embodiments of the present invention, sampling of the skin is performed in a minimally-invasive or non-invasive manner. Examples of methods that may be employed for skin sampling, include, but are not limited to: punch biopsy, scraping, tape stripping, or suction blistering.

The biomarkers which are tested for may be any biomarker or biomarkers which are known in the art. Biomarkers represent an up and coming field of research. The presence or absence of different biomarkers, as well as their relative quantities, may be indicative of a wide range of skin conditions, including, but not limited to, UV damage, aging, dryness, etc. . . . Thus, the present invention employs the use of biomarkers in order to obtain vital, clear, and specific information about the condition of the individual's skin who is being tested. The use of biomarkers allows for the precise and specific characterization of an individual's skin profile, based on real scientific information, not on speculation.

The unique biomarker profile produced is preferably compared and analyzed using a computerized biomarker database. This database is preferably formed through testing on a wide range of individuals, so as to produce a library of biomarker information and profiles. Also, an individual's biomarker profile may be compared to a biomarker profile of the same individual produced on a previous occasion, so to directly assess changes in an individual's skin conditions.

A personalized skin-care product is then produced, according to the analysis performed through comparison between the individual's biomarker profile and the biomarker database.

The biomarkers which are tested for preferably cover a very large range of possible skin conditions, both pathological, and physiological. Such skin conditions may include, but are not limited to: acne, atopic dermatitis, irritation, allergy, seborrheic, aging, dryness, baldness, low pH, UV damage, etc. . . . In this manner, the skin-care product provided is adapted for treating all of the conditions from which a person suffers. It is also noted that through the use of biomarkers, skin conditions may be detected which would otherwise go unnoticed due, for example, to their fairly undeveloped state. Thus, using the method of the present invention, skin conditions which may worsen in the future, are treated at an early state. The method of the present invention thus provides for a health-promoting and disease-preventing skin-care product.

It is further noted that the method of the present invention preferably uses a combination of specific active ingredients which are, each individually, already known for the treatment of specific skin conditions.

The skin-care product may be provided in any suitable form, such as, but not limited to, a gel, cream, or emulsion. A further unique feature of the present invention is that, if desired, the individual may specify certain preferences regarding the product provided. For example, the individual may specify that the composition be formed from only organic ingredients. The individual may also chose a specific aroma, fragrance, or color, for the composition to have.

The personalized approach has been developed over the past few years as a result of the understanding of high skin types deviation among the population. This had been accompanied by developing innovative technologies capable of identifying specific biomarkers, supporting a personalized diagnosis and treatments.

The inventors of the present invention have developed nano-chemical and bio-technologies to achieve an accurate matching of skin preparations to skin diseases and sub-pathogenic skin conditions in their individual context.

Non invasive methods (such as measurements of skin spectral fluorescence, RedOx state, cytokine expression, pH, metal contents, etc.), as well as minimal invasive skin sampling, (such as tape striping and epidermal sheet separation by suction blistering) support the characterization of a range of biological profiles, corresponding to various skin diseases. In parallel, nanotechnologies are used to develop optimized drug delivery systems (DDS).

A sophisticated tool is needed for statistical analysis of biomarker profiles associated with clinical conditions and with drug administration. Therefore, a bio-informatics data mining protocol is proposed, as well as biomarker profile analysis software. Individual data on patients' history, diagnosis and therapeutics is also considered for the development of a fully personalized skin treatment.

The computer-based decision support system (DSS) will assist dermatologists for diagnosis and prescription of more accurately fitted therapeutics.

In summary, the present invention proposes a novel generation of customized products, and services, to fit the patients' tailored needs, thus significantly improving the treatment efficiency for the benefit of the patients.

According to preferred embodiments of the present invention, the skin sampling and testing methods may also be selected from one or more of the following listed categories:

a. Classical skin dermatological data acquisition for testing for the following parameters: skin roughness, moisture (conductivity), pH, barrier functioning (TEWL), etc.

b. Surface extraction of skin-secreted molecules (low molecular weight antioxidants such as LMWA's, nitric oxide metabolites, cytokines) extracted in aqueous solution, in order to allow chemical or immunological characterization of extracted components, and measurement of overall reducing capacity by cyclic voltametry.

c. Direct fluorometric measurements performed in-vivo on skin samples. Skin auto-fluorescence is measured at different wavelengths in order to evaluate the amounts of tryptophan residues (an indicator of skin proliferation), of collagen and elastin cross links and of lipofuscin deposit (indicators of skin aging). Fluorometric measurement of the amounts of carotenoids (and possibly other chromophores) that are evaluated by Raman spectrometry or surface optical-fiber fluorometer also may be performed.

d. Epidermis samples are taken using the suction blistering technique, a minimally invasive method allowing the characterization of specific biochemical markers by ELISA or immuno-blots.

e. Oxidative stress has been shown to be a direct consequence of UV irradiation and also involved in the pathways of inflammatory diseases (Fuchs J, Zollner T M, Kaufmann R et al (2001) Redox-modulated pathways in inflammatory skin diseases. Free Rad Biol Med 30: 337-353). The skin redox status is assessed non-invasively by analysis of skin secreted LMWA's (ascorbic acid, α-tocopherol, uric acid) in superficial aqueous extracts, and of the overall reducing capacity of the same extracts (by cyclic voltametry). Skin carotenoid measurements are performed by direct optical readings on skin in vivo by Raman spectrometry or surface optical-fiber fluorometry. Serum LMWA's (uric acid, bilirubin, sulphydryls) and overall reducing capacity are analyzed from blood samples. From epidermis samples (suction blisters), protein oxidation, glycation and lipoxidation are investigated by immunodetection of protein-bound carbonyl groups, of advanced glycation end products (AGEs), and of 4-hydroxynonenal (HNE), respectively, in cellular extracts by ELISA tests or Western immuno blots. Epidermis autofluorescence is measured at 488/600 nm, to evaluate the possible accumulation of lipofuscin as a final product of protein lipoxidation and cross-linking.

f. Epidermal proliferation, which declines with aging and is expected to be modulated by pathological stress and inflammation is evaluated by direct measurements of skin auto-fluorescence in vivo at 295/340 nm.

g. Extracellular matrix protein alteration has been shown to be indicative of dermis decay and is associated with aging. Collagen degradation is activated by the JNK-dependent stress pathway and greatly accelerated in photo-aging. degradation products are monitored by radioimmunoassay in serum samples. Collagen and elastin cross linking are also involved in age and stress-related dermal alterations. They are measured by optical readings of skin auto-fluorescence in vivo.

h. Inflammatory response may be investigated at different levels. Nitric oxide (NO.) is produced in keratinocytes by the inducible form of NO. synthetase (iNOS), which is regulated by inflammatory stimuli via NFκB activation. iNOS expression is increased in inflammatory diseases including contact dermatitis and psoriasis (Portugal M, Barak V, Ginsburg 1, et al (2007) Interplay among oxidants, antioxidants, and cytokines in skin disorders: present status and future considerations. Biomed Pharmacotherapy (in press). Nitric oxide metabolites nitrite and nitrate are measured, using spectrophotometric methods: 1) in superficial aqueous skin extracts; 2) in serum samples. Then, inflammatory cytokines are monitored by specific immuno detection: 1) in superficial aqueous skin extracts TNFα, IL-1β, IL-8 and IL-10, major mediators of inflammatory processes, expressed in keratinocytes are determined; 2) in serum samples, a larger selection of cytokines are checked, including TNFα, IL-1, IL-6 and IL-8, which are induced by NFκB (a stress-activated transcription factor responsible for cellular senescence in keratinocytes) and IL-10 (TNFα and IL-1α are induced by skin irritants, and their production by keratinocytes plays an important role in the pathophysiology of contact dermatitis; L-10 is produced by keratinocytes after UV irradiation and inhibits contact hypersensitivity); 3) in soluble extracts from suction blister epidermal sheets, all cytokines quoted above are analyzed and possibly other markers (especially ICAM-1, a cell-adhesion molecule inducible on keratinocytes, essential to the initiation and evolution of localized inflammation processes in skin), depending on the availability of sufficient amounts of skin material.

i. UV stress and inflammatory response have been shown to be associated with the activation of various signaling pathways involving MAP kinases, like stress response, cell cycle and proliferation, senescence and apoptosis activation. The markers of these pathways are checked in epidermal sheet extracts prepared from suction blisters, by ELISA tests or Western immuno blots. These assays include: 1) From the MAPK pathway, involved in cell survival and proliferation: phosphorylated EGF receptor and ERK. 2) From the stress-activated protein kinase (SAPK) pathways: p38, JNK and c-Jun, component with c-Fos of the AP-1 transcription factor, which induces collagen degradation. 3) IκB, specific inhibitor of the NFκB transcription factor, which plays a key role in the cellular responses to UV and other inflammatory stimuli. 4) p21 and p16, two transcription factors controlling the complementary, p53-dependent and p53-independent pathways of cellular senescence in human keratinocytes. 5) Ki67, a classical marker of cell proliferation abundant in psoriatic lesions 6) Bax and Bcl-2, two markers of the “intrinsic” (mitochondrial) pathway, and Fas R, one of the heads of the “extrinsic” (death receptor-associated) pathway of apoptosis. 7 Heat shock proteins HSP70 and HSP27, which play a protective role in response to different kinds of stress. 8. TGF-β, which is involved in various pathways related to proliferation, differentiation, apoptosis and senescence. 9. Since differentiation markers are subjected to changes of expression pattern in skin disorders, the expression of involucrin, cytokeratin 6 and cytokeratin 10 is monitored.

j. Single-nucleotide polymorphisms (SNP's) associated with individual skin conditions, may be investigated in DNA extracted from the cellular fraction of blood samples, using the oligonucleotide DNA microarray technology.

k. Blood samples can also be analyzed in the following manners: 1) DNA is extracted from the cellular fraction, in order to analyze single-nucleotide polymorphisms by oligonucleotide micro-array technology; 2) sera is prepared and their contents in LMWA's, nitric oxide metabolites, cytokines, and collagen degradation products indicative of dermis alteration, are analyzed.

l. Reactive oxygen species (ROS) can be detected by EPR spectroscopy, after local application of nitroxide radicals; the distribution and metabolism of these nitroxide probes allow to determine skin permeability and the redox status of epidermis and dermis.

m. Heavy water (D2O) incorporation in keratins can be measured in vivo, allowing to measure skin turnover.

n. “exhaled breath condensate” analysis provides data on low-molecular

LMWAs and inflammation markers in the lower respiratory airways.

It will be appreciated that the aforementioned methods are all based on recent developments in dermatological and physiological research. As knowledge and understanding of the human body continues to grow, other methods will also be possible for providing comprehensive assessment of a patient's skin condition.

### DETAILED DESCRIPTION OF THE INVENTION

The present invention employs both non-invasive and minimally invasive protocols for skin sampling. Non-invasive skin measurements which may be used include: skin fluorescence spectra, antioxidant capacity, epidermal cytokine release, RedOx state, skin pH, metal content, skin topological roughness, and natural moisturizing conditions. Minimally invasive skin sampling methods include tape stripping, and suction blistering. It is to be appreciated that methods for skin sampling are continuously being developed and improved. The present invention is intended to encompass all such non-invasive and minimally-invasive methods which are known, and which are currently being developed. The above methods are used to test for the presence and/or quantity of one or more biomarkers.

The results of the skin sampling are then compared to known biological material from different sources from esthetic surgery donated skin to the semi-invasive epidermal suction blistering (which yields full-thickness epidermal sheets) and upper layers tape stripping (which yields successive layers of stratum corneum) or in situ protein extraction (for the measurement of cytokines, free radicals, etc) will be compared for yield, feasibility and patients' compliance. After correlation studies between physical and biochemical characteristics determined by non-invasive methods such as in situ fluorescence, in situ protein extraction, pH determination, corneometry, etc, multifunctional biomarker analysis on skin samples and on reconstituted skin models (supplied by StratiCell) will be performed to build skin biomarker maps.

In order to assess a multifunctional characterization of skin, providing a full specific “map” of individual skin condition and definition of physical/biochemical characteristics and biomarkers characteristic of skin conditions and pathologies, several assays have to be performed on skin samples:
expression levels of types I and III collagens
expression of Sp1, Sp3, C-Krox, NF-kB, AP-1, etc
production of MMP-1, 2, 3, 9
expression of IL-1β, 1L-6, TGF-β1, 2, 3 and their receptors.
signaling pathways: MAPk, JNK, Akt, Stats, etc. . . .
cell proliferation rate and expression of cell cycle proteins (p21, p27, cyclin D1)

The results will lead to a better understanding of the ECM alterations and their impact on ECM-cell interactions in these skin diseases, from which new treatment approaches will be established.

It is noted that the method of the present invention is essentially a novel and non-obvious combination of many known emerging technologies the discovery of biomarkers associated with different physiological and pathological conditions, the development of novel sampling means, and the development of better drugs and drug-delivery systems. The present invention utilizes all of these three fields in order to provide a personalized, user-specific product for the general public.

Human skin types display many variations due to genetics, age, lifestyle, environment, seasons etc. The regular ways to differentiate cosmetic and dermocosmetic products by skin type (i.e.: dry skin, very dry, oily, normal and combination skin) or by skin condition (i.e.: atopic, psoriatic, seborrehic, ichtiotic, acne, sensitivity, irritation etc.) are highly limited and do not reflect the endless skin variability of real life.

Based on human genome data, and on novel biotechnological methods for skin analysis, the present invention presents the development of personalized cosmetics, wherein the dermo-cosmetic products are specially formulated from A to Z or slightly adapted to fit each patient's skin, in order to improve skin health, radiance, and beauty.

The present invention employs novel technologies to evaluate the biomarkers of various skin conditions including skin senescence, differentiation, apoptosis, dryness, barrier functioning and more. The skin analysis is based on a skin sample taken from the patient by one of various methods.

The term ‘sampling’, as used in the present invention, means any in-vivo or ex-vivo protocol enabling sampling of a small portion of the skin, skin layers, or skin cells, and comprising inter alia the following techniques of skin sampling, biomarkers sampling: suction blistering, punch biopsies, blade scraping, tape stripping, etc. . . .

A plurality of laboratory tests may be used for biomarkers determination. These tests are selected, yet not limited to: intracellular tests, including especially various techniques in the field of micro arrays, such as high-density and low-density cDNA arrays, biochip, gen-arrays, proteomic arrays etc. . . . These tests may also include other methods of molecular and cellular biology, such as flow cytometry (e.g., FACS) and other technologies, 2D electrophoresis method, Western blot, ELISA and other immunological techniques. Those methods have been proved useful, reliable, reproducible and cost-effective for skin analysis and biomarker determination.

The purpose of ex-vivo sampling techniques is to evaluate the effects of topical applications of various products on “cellular age” in epidermal cells. The evaluation is mainly provided by comparing transcriptional patterns of treated versus untreated portions of epidermis, or by comparing data from a later skin specimen with those from early ones. Additionally or alternatively, analysis is made of protein markers of ageing, and of age-related enzyme activities in epidermal samples from treated versus untreated skin portions, or from young versus old skin portions.

The dermatologic punch used in the present invention is preferably a round knife ranging from 2 to 10 mm in size. A plurality of 5 mm punches generally provides adequate epidermis samples for analysis. The experiment was found to be especially effective in healthy volunteers (i.e., patients) ages 18-65, without skin diseases or chronic medication or drug use who were not participating in any other research. Before performing a punch biopsy, the skin tension lines are determined. Thus, the skin is stretched perpendicularly to these tension lines before the incision, and an elliptic wound is formed in such a manner that it can be closed with the help of normal skin tension without “dog ears”. After cleansing the skin, local anesthesia is achieved by intradermal injection. The punch is placed perpendicularly to the skin surface. After applying a gentle pressure, it is rotated back and forth while advancing on the hub. The edge of the specimen is then grasped with toothed forceps, or “scooped” out with the punch, so as not to crush the skin. If the underlying fatty tissue retains the base of the sample, iris scissors are used to snip the tissue free. Hemostasis is obtained by applying aseptic solution, gauze and pressure. Subsequently, the wound is closed by one or two sutures to avoid a depressed scar.

Biopsies of skin samples are stored in “RNA later” solution (Promega) before RNA extraction, or put in PBS and frozen for protein analysis by ELISA or western blots. Total RNA is extracted using the “SV Total RNA Isolation System” (Promega). PolyA+ messenger RNA can be extracted using Micro-FasTrack mRNA Isolation Kit (Invitrogen). Two to four 5 mm-diameter biopsies are hence taken from each forearm, corresponding to 0.2 cm2 of epidermis per punch biopsy. About 100,000 keratinocytes are obtained from each punch biopsy, mixed with a small number of dermal fibroblasts (up to 5%). The yield of total RNA is about 10 mu.g per punch biopsy (up to 80 .mu.g per operation).

It is also in the scope of the present invention to provide another epidermal skin sampling protocol, namely tape-stripping. This protocol is useful for evaluation of the effects of topical applications of various products on “cellular age” in epidermal cells: analysis of protein markers of ageing, and of age-related enzyme activities, in treated versus untreated skin portions. Here again, healthy volunteers are selected. The tape-stripping experiments are performed on the flexor forearm. Adhesive tape is applied to the skin of the flexor forearm, pressed to the skin area with a roller and pulled off in one quick movement as described. The first tape strip is discarded. The next 5 to 20 tapes can be subjected to protein extraction for western blot analysis or enzyme assay. Wounds are dressed with special hydrocolloid dressing, and heal within one week with no residual scars. In this protocol, the extraction yields are low when compared with surgical methods, since only a minority of epidermal cells is removed from the skin. For a treated surface of 10 cm.sup.2, yields of 100-200 .mu.g of protein can be obtained, depending on the number of successive strips.

Alternatively, another epidermal sampling protocol was used, namely suction blisters technique. This technique is especially useful for analysis of transcriptional changes in the expression of ageing markers, in skin epidermis from young and old donors, using high-density cDNA micro-arrays. From this data, determination of a set of markers showing differential transcription patterns (“profiles”) is provided, especially useful for evaluation of the effects of topical applications of various products on “cellular age” in epidermal cells, carried out by comparing transcriptional patterns in treated versus untreated portions of the epidermis.

Accordingly, metal cups are attached to each forearm skin surface. Skin is suctioned using a vacuum pump with negative pressure of 150 mm Hg. Epidermal blisters, 1.6 cm in diameter, are induced after 2-5 hours of suction. Blister fluid is collected for protein analysis by ELISA or Western blots. Epidermis samples are cut out from blister roofs and immediately frozen in liquid nitrogen, or processed right away for RNA extraction using the “SV Total RNA Isolation System” (Promega). Total RNA can be analyzed using the “Affymetrix cDNA micro arrays” transcript screening system or by specific skin low-density gene-arrays. Alternatively, epidermis samples are processed for analysis of specific protein markers, protein modification, or enzyme activities. Wounds are dressed with special hydrocolloid dressing, and heal within 10 days with no residual scars. Two to four blisters are suctioned on each forearm. About 2 cm.sup.2 of epidermis and about 106 cells, essentially keratinocytes, can be obtained from each blister. About 100 .mu.g of RNA or 500 .mu.g of protein may be extracted per blister.

It was found that the yields of the hereto-defined methods (namely punch biopsies, tape stripping and suctions blisters) retained for systematic sampling provide sufficient amounts of RNA or proteins for at least one test per patient (in the case of punch biopsies) or more. Tape stripping is restricted to the study of protein modifications. Suction blistering was found appropriate for all the purposes of this project, including immunofluorescence and enzyme assays in situ. Because punch biopsies are commonly practiced in most dermatology departments, this method provides a valuable alternative for obtaining RNA samples whenever dependence on a specialized hospital facility may be a limiting factor.

It is further acknowledged that epidermis sampling is needed for the analysis of age-sensitive RNA transcripts and protein biomarkers, in order to evaluate the biological effects of potential active anti-ageing components following topical application.

It is additionally acknowledged that by performing periodical skin sampling and analyzing RNA extracts from skin one may determine up or down levels in the transcriptions of some ageing gene biomarkers.

Punch biopsy is the most direct method, allowing immediate sampling of skin slices that can be processed for either RNA or protein extraction. However, this method is affected by several drawbacks: a. local anesthetic is necessary, and might interfere with the expression of short-lived transcripts; b. the wound is expected to leave a minor scar, making the other methods preferable whenever punched slices of skin are not specifically needed; and c. punched skin slices contain dermis, and keratinocyte separation is delicate because of the scarcity of the material. This inconvenience is limited for RNA sampling because the accompanying dermis is constituted mainly of extracellular matrix and can contribute only a minority of cells and of RNA (less than 10%); but the interference of dermis is more severe for protein sampling; therefore the use of punch biopsies has to be restricted to RNA analysis. For cosmetic purposes, it is important indeed that all tests address “upper skin layers” (i.e.: epidermis) only, and do not penetrate to sample deeper layers of the skin, in order to remain legally in the field of cosmetology.

Tape stripping is much less invasive than punch biopsy, leaving only superficial wounds that heal without residual scars. Even after 30 repeated strippings, only epidermal material is collected, but the cells are heterogeneous and mostly representative of upper layers. These are keratinocytes, more or less deeply engaged in terminal differentiation and cell death, with very low yields of transcripts. Were a transcriptional pattern to be detectable, it would represent terminal differentiation rather than ageing status. The same applies also to protein markers, except protein modifications, which are expected to be conserved in cellular proteins along the differentiation process. Tape stripping therefore seems to be the method of choice for the study of protein modifications in epidermis.

Skin scraping with a scalpel or razor blade resembles tape stripping, but is more traumatic, and inaccurate in the selection of cell layers. It has therefore not been considered.

Suction blistering offers the advantage of performing a clean separation between dermis at blister bottom and epidermis at blister roof. Epidermis comes off in its entirety including the basal layer. The procedure is almost painless, no anesthetic is used, and the wound is superficial and heals without a scar. Blister roof, when cut, can be processed directly either for RNA or for protein extraction. Blister fluid can be collected and used for protein analysis. Suction blistering appears therefore most appropriate for our purpose, yet has the drawback that suction itself has to be continued for 2 to 4 hours at ambient temperature, during which the marker profiles might be perturbed.

It is also in the scope of the present invention to use cellular markers for flow cytometry. Those non-immunologic, cellular markers are selected in a non-limiting manner from the group of biomarkers characterized by: .beta.-galactosidase activity, preferably studied by means of an intracellular assay; proteasome chymotrypsin-like and postglutamyl-peptidase activities, preferably studied by means of an intracellular assay; total protein content, preferably studied by FITC labeling. Some of those markers are characterized by an expected up-regulation upon ageing, and others by down-regulation. Beta.-galactosidase activity was found to increase notably upon ageing in most mammalian tissues including epidermis. More specifically, it was proved that beta.-galactosidase activity increases by a factor 4 in human epidermis between the ages of 20 and 80.

It is also in the scope of the present invention wherein the aforementioned undirected biomarkers are selected from Apo J (i.e., clusterin), Ki-67, carbonyl groups e.g., (protein oxidation), 4-hydroxy-2-nonenal (i.e., lipid peroxidation adduct in proteins), carboxymethyl-lysine or advanced glycated endproducts (e.g., at least two kinds of glycoxidation adducts in proteins), ubiquitin adducts on proteins, total amount of cellular proteins or any combination thereof.

It is acknowledged in this respect that the amount of ApoJ transcripts (i.e., an mRNA) increases, upon replicative senescence, by a factor 11 in human osteoblasts and by a factor of 24 in rat embryonal fibroblasts. Total protein contents in human keratinocytes were found to increase by at least a factor of 5 upon replicative senescence.

It is also in the scope of the present invention wherein enzymatic biomarkers are selected from .beta.-galactosidase, proteasome chymotrypsin-like or postglutamyl-peptidase activities. Alternatively or additionally, the biomarkers involved in a senescence pathway may be selected from: p161NK4a; Cdk4; Cdk6; p19ARF; p53; MD2; p21Cip1; PTEN; p27Kip1 or PI 3-kinase. Alternatively or additionally, the involved biomarkers in apoptosis pathways are selected from: Fas, Fas L, FADD, activated caspase 8 (i.e., cleaved protein), p53, Bcl-2, Bax, Apaf-1, caspase 3 activity, annexin V-binding sites.

The amounts of p161NK4a were found to increase by at least a factor of 2 upon replicative senescence in cultured human keratinocytes. The amounts of Fas receptor were found to increase by a factor of 4 in human epidermis between the ages of 20 and 80.

It is also in the scope of the present invention wherein enzymatic biomarkers involved in keratinocyte differentiation are selected from involucrin and from cytokeratines.

The proteasome chymotrypsin-like and postglutamyl-peptidase activities, in human epidermal extracts, were hereto proved to decrease by a factor of 3 between the age of 20 and the age of 60-70.

It is appreciated that the biomarkers which are tested for may comprises any predetermined set of biomarkers, and the above descriptions were provided for the purposes of example only.

1. A method for providing a personalized skin-care composition for an individual, comprising;
(a) sampling the skin of an individual for whom a personalized skin-care composition is to be provided;
(b) testing for a predetermined set of biomarkers so as to produce an individual biomarker profile;
(c) analyzing said biomarker profile using a biomarker profile database;
(d) producing a personalized skin-care composition based on the results of step (c);
wherein said personalized skin-care composition is adapted for treating one or more skin conditions which the individual has.

2. The method of claim 1, wherein said skin condition is selected from at least one of the group consisting of: psoriasis, atopic, seborrheic, ichtiotic, acne, xerotic, irritation, and allergy.

3. The method of claim 1, wherein said skin condition is selected from at least one of the group consisting of: ageing, dryness, hyperesthetic, hyperpilose, baldness, poor cicatrizing, and high pH.

4. The method of claim 1, wherein said sampling the skin is performed based on a method which is selected from the group consisting of: punch biopsy, scraping, tape stripping, and suction blister.

5. The method of claim 1, including protein analysis selected from the group consisting of: ELISA assays, electrophoresis, Western immuno blotting, or any combination thereof.

6. The method of claim 1, wherein said biomarkers are related to at least one pathway selected from the group consisting of: cellular senescence, apoptosis, and cell differentiation.

7. The method of claim 1, wherein said biomarkers are selected from the group consisting of: Apo J. Ki-67, carbonyl groups, 4-hydroxy-2-nonenal, carboxymethyl-lysine or advanced glycated endproducts, ubiquitin adducts on proteins, total amount of cellular proteins or any combination thereof.

8. The method of claim 1, wherein said biomarkers are selected from the group consisting of: .beta.-galactosidase, proteasome chymotrypsin-like or postglutamyl-peptidase activity.

9. The method of claim 1, wherein biomarkers involved in a senescence pathway are selected from the group consisting of: p16INK4a; Cdk4; Cdk6; p19ARF; p53; MDM2; p21Cip1; PTEN; p27Kip1 or PI 3-kinase.

10. The method of claim 1, wherein said biomarkers are involved in apoptosis pathways and are selected from the group consisting of: Fas, Fas L, FADD, activated caspase 8 (cleaved protein), p53, Bcl-2, Bax, Apaf-1, caspase 3 activity, or annexin V-binding sites.

11. The method of claim 1, wherein said biomarkers are involved in keratinocyte differentiation are selected from involucrin and from cytokeratines.

12. The method of claim 1, wherein said composition is in a form selected from the group consisting of: a cream, emulsion, or gel.

13. The method of claim 1, further comprising comparing said biomarker profile to a previous biomarker profile for the same individual.

14. The method of claim 1, wherein said producing a personalized skin-care composition includes taking into account personal preferences of the individual for whom the composition is being made.

15. The method of claim 1, including surface extraction of skin-secreted molecules, and measurement of said molecules' overall reducing capacity.

16. The method of claim 1, including direct fluorometric in-vivo measurement of skin samples.

17. The method of claim 1, including assessing the redox status of said skin.

18. The method of claim 1, including determining epidermal proliferation by measurement of skin auto-fluorescence.

19. The method of claim 1, including determining extracellular matrix protein alteration.

20. The method of claim 1, including investigating the inflammatory response of said skin.

21. The method of claim 1, including measuring for markers involved in UV stress and inflammatory response.

22. The method of claim 1, including blood sampling and DNA extraction for the analysis of single-nucleotide polymorphisms.

23. The method of claim 1, including detecting reactive oxygen species.

24. The method of claim 1, including measuring heavy water incorporation in keratins.

25. The method of claim 1, including testing exhaled breath condensate for providing data on low molecular LMWA's and inflammation markers.
